(19) United States
(12) Patent Application Publication
(10) Pub. No.: US 2011/0098756 A1
(43) Pub. Date: Apr. 28, 2011

(54) PEDICLE SCREW

(76) Inventor: James K. Brannon, Leawood, KS (US)

(21) Appl. No.: 12/986,064

(22) Filed: Jan. 6, 2011

Related U.S. Application Data
(63) Continuation of application No. 12/369,388, filed on Feb. 11, 2009, now abandoned.

(51) Int. Cl. A61B 17/86

(52) U.S. Cl. 606/309

(57) ABSTRACT

The present invention provides an improved orthopedic fastener adapted for receipt within a receiving surface associated with an orthopedic material, the fastener comprising a head separated from a tapered end along a threaded shaft, the threaded shaft having at least one helical thread extending therebetween, at least one region of irregularity positioned along the helical thread and presenting an axis of retention, the axis of retention limiting longitudinal movement of the threaded shaft from the receiving surface.

### FIELD OF THE INVENTION

This invention relates generally to orthopedic fastening devices. More specifically, the present invention relates to a threaded fastener adapted for insertion along a compromised bone region, the threaded fastener having a head and a shaft with plural alignment structures adapted for engagement by surrounding material.

### BACKGROUND OF THE INVENTION

In general, orthopedic fasteners, also referred to as pedicle screws, are represented as screw fasteners having a screw shaft and a rotatable head for rotationally inserting the threaded fastener into bony material or into a receiver specifically adapted for receiving these threaded fasteners. However, many of these receiving surfaces are treated to limit the interaction between the shaft or receiver and the surrounding material. Often these fasteners are metallic and fabricated from steel, cobalt-chrome and titanium in pure and/or alloy form. To limit the tissue response or biological interaction of these materials, the surface of the orthopedic fastener is generally modified by polishing the fastener using an abrasive material such as alumina, sand or ceramic material. Alternatively, the some fasteners are modified using electrical or chemical etching processes to polish the fastener surface, limiting interaction between the surrounding material and the fastener. However, these known orthopedic fasteners may be rejected by the body for various reasons. Some orthopedic fasteners which also present a generally smooth or non-reactive surface rely upon mechanical interlocking surfaces to secure the fastener in place. Over time these interlocking surfaces can weaken or fail. In addition, the time necessary for regeneration and/or repair of the surrounding material may be excessive when using orthopedic fasteners with smooth surfaces.

Additionally, some prior art fasteners utilize a threaded fastener having a head and an axis of rotation aligned with a threaded shaft. When it is desired to loosen the fastener, the fastener is typically rotated in a counter clockwise direction using a reverse rotational force. Generally, the force is transmitted from a rotational device to the head of the fastener along the axis of rotation. The torque necessary to loosen or remove the fastener generally depends on the strength of the threaded shaft in connection with the surrounding material. However, using orthopedic materials, which may be weak or damaged, these connections may not be as strong as desired.

Therefore, there exists a need for an orthopedic fastener which securely fastens to the surrounding orthopedic material along an axis of retention, while encouraging the regeneration of the surrounding material.

Several prior art attempts to address these concerns themselves present disadvantages which are addressed by the present invention, namely the present invention, a orthopedic fastener with regions of irregularity and an axis of retention for securing the fastener in place. For example, some prior art attempts include fabricating an orthopedic fastener from biodegradable material which over time dissolves. In addition, some prior art attempts include installing a separate fastening device within the boney material and some prior art attempts include fabricating orthopedic fasteners from non-reactive expensive metals for strength.

Each of these systems leaves a need for simple reliable orthopedic fastener and method for securing surrounding orthopedic material using a threaded fastener with a head extending towards a threaded shaft, the threaded shaft presenting an axis of connectivity between the threaded fastener and the surrounding material.

### SUMMARY OF THE INVENTION

The present invention reduces the difficulties and disadvantages of the prior art by providing an improved orthopedic fastener adapted for receipt within a receiving surface associated with an orthopedic material, the fastener comprising a head separated from a tapered end along a threaded shaft, said threaded shaft having at least one helical thread extending therebetween, at least one region of irregularity positioned along the helical thread and presenting an axis of retention, and said axis of retention limiting longitudinal movement of the threaded shaft from the receiving surface.

### BRIEF DESCRIPTION OF THE DRAWINGS

FIG. 1 is a front perspective view of the orthopedic fastener according to the present invention.

FIG. 2 is a top plan view of the orthopedic fastener illustrated in FIG. 1.

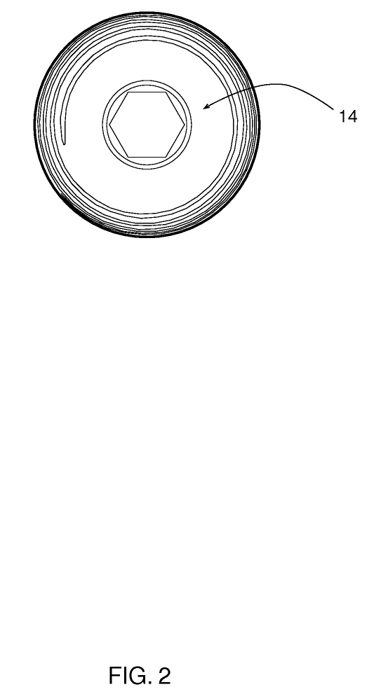

FIG. 3 is a bottom plan view of the orthopedic fastener illustrated in FIG. 1.

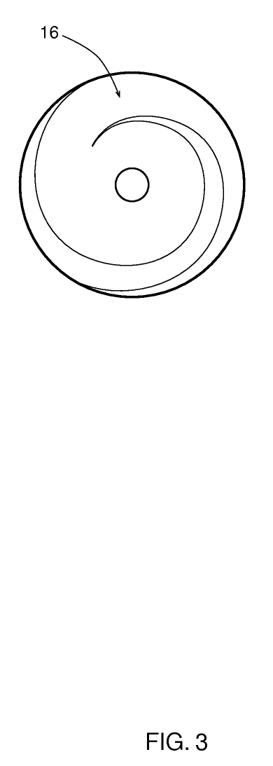

FIG. 4 is a cross sectional elevational view of the orthopedic fastener illustrated in FIG. 1.

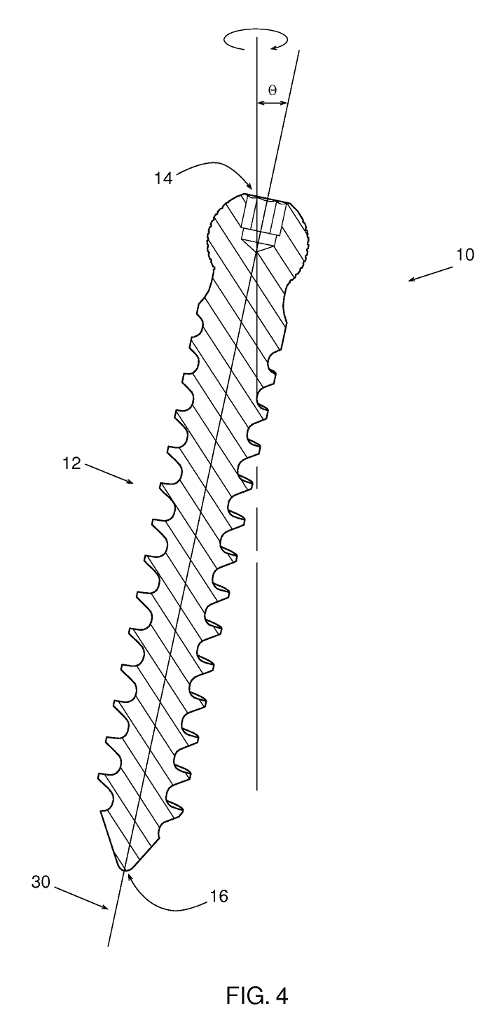

FIG. 5 is a magnified sectional view of a helical thread associated with the orthopedic fastener taken along lines A-A in FIG. 1

FIG. 6 is a front perspective view of the orthopedic fastener according to an alternative configuration of the present invention.

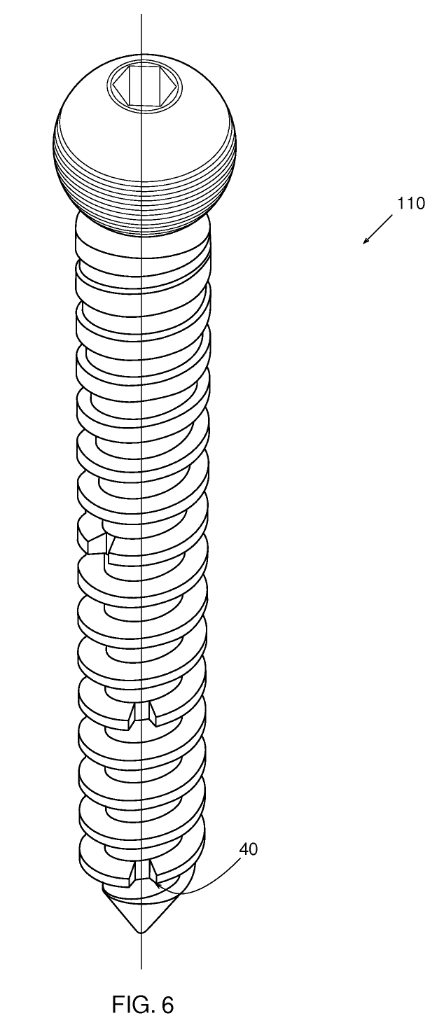

### DETAILED DESCRIPTION OF THE INVENTION

### I. Introduction.

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

### II. Orthopedic Fastener.

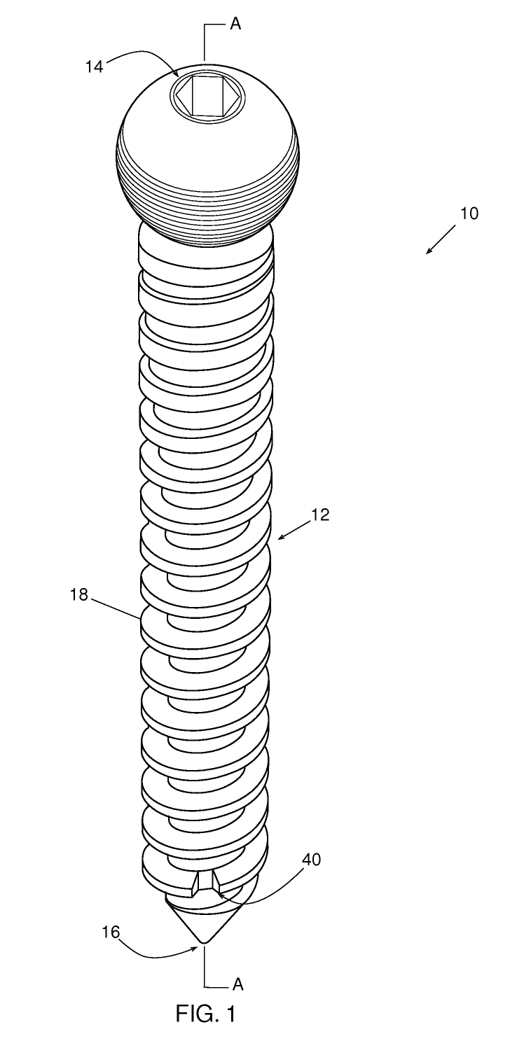

Referring to FIG. 1, an embodiment of the present invention, an improved orthopedic fastener is generally indicated by reference numeral 10 having a threaded shaft 12 extending between a head 14 and a tapered end 16, the threaded shaft 12 including a spiral or helical thread 18 encircling the shaft. It is understood that the fastener 10 embodying the present invention may have substantially any head 14 configuration without departing from the spirit and scope of the present invention.

An axis of retention 30 is depicted in FIG. 1. Generally, the orthopedic fastener 10 is secured to surrounding material (not shown) upon insertion of the orthopedic fastener 10 into a receiving surface (also not shown), such as the surrounding orthopedic material. The spiral or helical thread 18 generally extends continuously from the head to the tapered end 16 of the orthopedic fastener 10, the helical thread 18 having a generally constant diameter portion in relation to the threaded shaft 12 and a pitch 20 defined therebetween. In this way, the orthopedic fastener 10 is used for fixation of the orthopedic material.

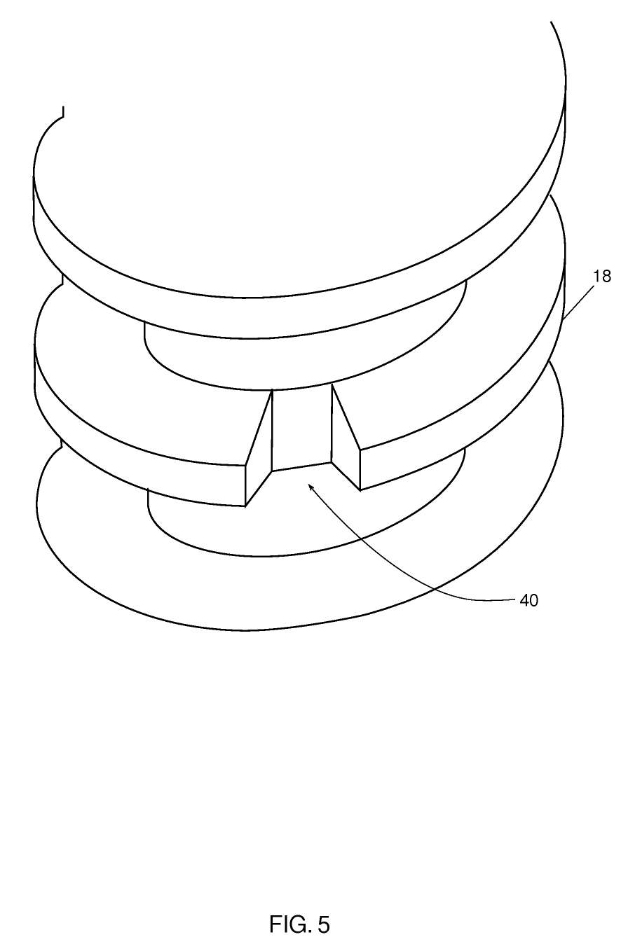

Additionally, the orthopedic fastener 10 includes at least one region of irregularity 40 which is represented as a slot arranged normal to the helical thread 18. The region of irregularity 40 is further illustrated in FIG. 5, as a notched slot extending from a proximate end associated with the outer surface of the helical thread 18 inwardly towards the distal end associated with the threaded shaft 12. The illustrated notched slot 40 has a tapered orientation with the proximate end being spaced further apart than the distal end, the notched slot 40 being wider near the outer surface and narrower near the threaded shaft 12. In general, the regions of irregularity 40 interact with the surrounding orthopedic material at the notched slot 40 which interconnects to the surrounding material. In addition, the notched slot 40 has a leading and trailing edge 22, 24 which are generally angularly orientated with respect to each other. Alternatively, the regions of irregularity 40 may be include, but are not limited to bumps, ridges, porous or other similarly irregular surfaces spaced along the helical threads 18. While the arrangement of the regions of irregularity 40 may vary, generally, they are configured as notched slots positioned circumferentially along the helical thread 18 at predetermined locations depending on a desired axis of retention 30.

Without limiting the application of the present invention, the improved orthopedic fastener can be applied to both human and animal patients and subjects in connection with a wide variety of medical, dental and veterinary conditions and treatments. For example, the orthopedic fastener 10 may be used in replacement, repair of diseased, damaged or worn surfaces and various soft tissue injuries. Generally, the regions of irregularity 40 are adapted for intermediate connectivity with orthopedic materials or surfaces, tendons or separated tissue at the subcutaneous layer of the patient by placing the region of irregularity 40 against the orthopedic material, tendons or soft tissue portions and bonding them together utilizing tissue interdigitation generally at the leading and trailing edges 22, 24. Alternatively, the leading and trailing edges 22, 24 can be enhanced to facilitate and/or enhance tissue interdigitation.

Initially the orthopedic fastener 10 is rotationally threaded at least partly through the receiving surface into the orthopedic material. After receipt of the orthopedic fastener by the receiving surface, unexpectedly, the surrounding material is angularly secured to the regions of irregularity 40. The axis of retention 30 is generally defined in part by the alignment of the regions of irregularity 40 along the helical thread 18, the axis of retention 30 limiting longitudinal movement of the orthopedic fastener 10. Depending on the positioning of the regions of irregularity 40, the axis of retention 30 may vary, extending angularly from an axis of rotation 32. During insertion of the orthopedic fastener 10, the axis of retention 30 may be similar to the axis of rotation 32 for threaded receipt of the orthopedic fastener 10 by the receiving surface. However, during removal, the regions of irregularity 40 are adapted for providing an angular offset of the axis of retention 30 from the axis of rotation 32. The offset may be at least partially defined by the spacing between plural regions of irregularity 40 asymmetrically along the helical thread 18. As additional regions of irregularity 40 are positioned asymmetrically along the helical threads 18, the axis of retention 30 may be greater or lesser with respect to the axis of rotation 32. In this way, the axis of retention 30 may impact the torque and angle of rotation necessary to remove the orthopedic fastener 10 from the surrounding material.

Additional benefits may be realized by axially orienting the region of irregularity 40 including defining a passageway through the orthopedic fastener 10 for the removal of surrounding material debrided during rotational receipt of the improved orthopedic fastener 40 by the receiving surface. The passageway associated with the regions of irregularity 40 also provides an inlet for the receipt and transmission of orthopedic materials into the receiving surface or surrounding material along the orthopedic fastener 10 to enhance the fixation of the orthopedic fastener 10 or for repairing the surrounding orthopedic material.

Additional the regions of irregularity 40 provide for easier and more secure rotational insertion of the orthopedic fastener 10 by the receiving surface while facilitating improved repair and connectivity of the surrounding tissue with the orthopedic fastener. In contrast to the traditional method of utilizing non-reactive orthopedic fasteners with smooth surfaces, the present invention actually improves repair and connectivity between the orthopedic fastener, the receiving surface and other the surrounding material.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

Having thus described the invention, what is claimed as new and desired to be secured by Letters Patent is as follows:

1. An improved orthopedic fastener adapted for receipt within a receiving surface associated with an orthopedic material, the fastener comprising:
a head separated from a tapered end along a threaded shaft, said threaded shaft having at least one helical thread extending therebetween,
at least one region of irregularity positioned along the helical thread and presenting an axis of retention, and
said axis of retention limiting longitudinal movement of the threaded shaft from the receiving surface.

2. The improved orthopedic fastener of claim 1 wherein said region of irregularity is adapted for intermediate connectivity with surrounding surfaces

3. The improved orthopedic fastener of claim 1 wherein said region of irregularity further comprises a leading edge and a trailing edge, said leading and trailing edges being angularly oriented with respect to each other.

4. The improved orthopedic fastener of claim 3 wherein said leading and trailing edges are adapted to facilitate tissue interdigitation.

5. The improved orthopedic fastener of claim 1 wherein said region of irregularity provides a passageway through said orthopedic fastener.

6. The improved orthopedic fastener of claim 1 further comprising plural regions of irregularity asymmetrically spaced along said helical thread.

7. The improved orthopedic fastener of claim 1 further comprising a rotational axis normal to the receiving surface about which said head is rotated wherein said rotational axis is angularly offset from said axis of retention.
